(19) United States
(12) Patent Application Publication
(10) Pub. No.: US 2022/0338480 A1
(43) Pub. Date: Oct. 27, 2022

(54) ANTIMICROBIAL COMMON TOUCH SURFACES

(71) Applicant: JNT TECHNOLOGIES, LLC, Salt Lake City, UT (US)

(72) Inventors: Giridhar THIAGARAJAN, Salt Lake City, UT (US); Sivaprasad SUKAVANESHVAR, Salt Lake City, UT (US)

(73) Assignee: JNT TECHNOLOGIES, LLC, Salt Lake City, UT (US)

(21) Appl. No.: 17/633,986

(22) PCT Filed: Aug. 7, 2020
(86) PCT No.: PCT/US2020/045398

§ 371 Date: Feb. 9, 2022

Related U.S. Application Data
(60) Provisional application No. 62/884,911, filed on Aug. 9, 2019.

(51) Int. Cl. A01N 59/20; A01N 59/16; A01P 1/00

(52) U.S. Cl. CPC A01N 59/20; A01N 59/16; A01P 1/00

(57) ABSTRACT

An antimicrobial device can include a common touch surface of a non-metallic material, and an antimicrobial metal layer applied to the common touch surface at an average thickness ranging from a single metal atom in thickness to 1 mm.

### BACKGROUND

In our daily lives we touch many non-metallic surfaces, e.g., polymer (rubber, plastic, styrofoam), wood, leather, ceramic, porcelain, glass, fabric and textiles, paper, carbon fiber, etc., such as transportation handles, e.g., bike handles, scooter handles, motorcycle handles, automobile steering wheels and gear shifters; other types of handles, e.g., door handles, door knobs, gym equipment handles, bathroom fixture handles; as well as other plastic objects that regularly come in contact with germs, e.g., plastic credit cards, trays, water bottles, shoes, cell phones, chairs (baby high chairs, chairs in public places, etc.), tables, arm rests, etc. Such objects and surfaces are often shared among (and touched by) multiple individuals, or repeatedly by the same individual. When touching a non-metallic surface, for example, a certain microbial load transfers from the user to the non-metallic surface, posing a risk of contamination or transfer to a subsequent user. This is particularly problematic for polymer surfaces because these materials often are carbon and/or silicon rich materials that can provide a refuge for invading microbes due to their organic or organic-like structure and also their lack of self-protective properties. As a result, the prevalence of polymeric common touch surfaces represents a public health risk, particularly during flu season or when other communicable diseases, e.g., COVID-19, may be present and may be transmittable from one person to another via the common touch surface.

### BRIEF DESCRIPTION OF THE DRAWINGS

FIG. 1A schematically illustrates an example scooter with common touch surfaces in the form of polymer hand grips coated or plated with an antimicrobial metal layer in accordance with the present disclosure;

FIG. 1B schematically illustrates an example arm rest for a chair with common touch surfaces in the form of a plastic substrate partially coated or plated with an antimicrobial metal layer in accordance with the present disclosure;

FIG. 1C schematically illustrates an example toilet seat with common touch surfaces in the form of a plastic top coated or plated with an antimicrobial metal layer in accordance with the present disclosure;

FIG. 2 is a bar graph depicting the antimicrobial performance of multiple copper-plated polymers in terms of Log-scale reduction of Staphylococcus aureus compared to otherwise identical uncoated polymers;

FIG. 3 is a bar graph depicting the antimicrobial performance of a copper-plated silicone in terms of Log-scale reduction of Staphylococcus epidermidis and Staphylococcus aureus compared to an otherwise identical uncoated silicon;

FIG. 4 is a bar graph depicting the antimicrobial performance of example copper-plated polymeric hand grips compared to uncoated polymeric hand grips against Staphylococcus aureus;

FIG. 5 is a bar graph depicting the antimicrobial performance of an example copper-plated toilet seat compared to an uncoated plastic toilet seat against Staphylococcus aureus;

FIG. 6 is a bar graph depicting the antimicrobial performance of an example copper-plated toilet seat compared to an uncoated plastic toilet seat against Pseudomonas aeruginosa; and

FIG. 7 is a bar graph depicting the antimicrobial performance of an example copper-plated toilet seat compared to an uncoated plastic toilet seat against Candida albicans.

### DETAILED DESCRIPTION

One approach to providing antimicrobial properties to a polymeric common touch surface is to coat a surface of the polymeric common touch surface at a location susceptible to infection, such as bacterial colonization. One of the technical challenges with many antibiotic coatings, however, is their lack of long term effectiveness. For example, drug-eluting coatings often deplete quickly due to limited availability of a drug reservoir after which they can be ineffective, e.g., a thin coating does not provide a large reservoir, and on the other hand, non-eluting passive coatings with use tend to be less resistant after which they themselves harbor microbes fairly quickly. This can limit the antimicrobial effect of such technologies to only a few hours, a few days, but typically not in the order of weeks. As long term devices, e.g., 5 days to 5 years, 1 week to 2 years, 2 weeks to 2 years, 4 weeks to 2 years, 1 week to 1 year, 2 weeks to 1 year, 4 weeks to 1 year, 1 week to 6 months, 2 weeks to 6 months, 4 weeks to 6 months, etc., would benefit greatly from a more durable and/or longer lasting form of antimicrobial protection.

Thus, the present disclosure is drawn to the application of a continuous antimicrobial metal layer to a polymeric common touch surface. The antimicrobial elemental metal coating or metal alloy coating can be deposited on the polymeric common touch surface in a non-eluting fashion. If a metal alloy, the metal alloy can include at least one metal at a high enough concentration to exhibit antimicrobial properties. As an example, an antimicrobial metal coating can be applied by electroless deposition or other technique, such as sputtering, spraying, weaving, dip coating, etc. However, rubbers, plastics, and other polymers can be effectively coated reliably and at a practical thickness using electroless deposition or other electroplating techniques. These coating technologies can be particularly useful in applications where the antimicrobial metal to be applied may be selected to be a non-leaching metal, such as the contact killing metal copper. In further detail, though any antimicrobial metal can be used, such as silver, zinc, or gold, or alloys thereof, in one specific example, the antimicrobial metal can be or includes a contact killing metal, such as copper.

In accordance with this, the present disclosure is drawn to an antimicrobial device, which can include a common touch surface of a polymeric material, and an antimicrobial metal layer applied to the common touch surface at an average thickness ranging from a single metal atom in thickness to 1 mm. In one example, the antimicrobial metal layer is not applied to areas of the device that are not at the common touch surface, but can be applied elsewhere as well in some examples. The antimicrobial metal layer can be applied as an electroplated elemental metal or metal alloy layer positioned on the common touch surface. For example, the antimicrobial metal layer can include elemental copper, elemental silver, elemental zinc, elemental gold, or an alloy thereof. The antimicrobial metal layer in one specific example is elemental copper or an alloy of elemental copper, as copper is a good microbial contact killer. The antimicrobial device can be, for example, a public use device. In another example, the common touch surface is on a hand grip, hand hold, or a hand actuator, and the common touch surface can include a location designed for repeatable contacted by a hand of multiple hosts, or even multiple uses by the same host. In some examples, the antimicrobial metal layer can be an electroplated metal having a thickness from 0.0001 μm to 50 μm, or can have a thickness from 0.001 μm to 0.1 μm, for example.

In another example, a method of reducing the spread of microbes from host to host (or multiple uses by the same host) can include providing the antimicrobial device described above, e.g., having a common touch surface of a polymer material having thereon an antimicrobial metal layer. The method can further include using the antimicrobial device with a first host coming into skin contact with the common touch surface, and subsequently using the antimicrobial device with a second host coming into skin contact with the common touch surface at a later point in time. In between the first hosing using the antimicrobial device and the second hosting subsequently using the antimicrobial device, the antimicrobial metal layer in this example\kills a portion of the microbes thereon left by the skin contact by the first host. Stated another way, the method can include initiating a use of the antimicrobial device by a first host coming into skin contact with the common touch surface, and then initiating a subsequent use of the antimicrobial device. The subsequent use includes a second host (different than the first host) or the same host coming into skin contact with the common touch surface. In between the use and the subsequent use, the antimicrobial metal layer kills a portion (which includes some or all) of the microbes thereon that were left by skin contact by the first host. The antimicrobial device can include, for example, a common touch surface with an interface for skin contact using the hand or other skin surface, and the skin contact by the first user is by using the hand (or other skin surface) and the skin contact by the second user is also by using the hand (or other skin surface). The antimicrobial device can be included, for example, on a rental device, and the common touch surface is included as part of a hand grip, a hand actuator, a hand hold, or a hand rest. The antimicrobial device can likewise be included on a public transportation device, and the common touch surface can be included as part of a hand grip, a hand actuator, a hand hold, or a hand rest. In another example, there may be instances where a skin surface of a common type is touched repeatedly by multiple hosts, or the same host over and over again, that is not the hand, e.g., a copper plated insole may be used where the feet commonly touch a surface.

In another example, a method of manufacturing the antimicrobial device described herein can include applying the antimicrobial metal layer to the common touch surface of the polymeric material to form the antimicrobial device. Applying the antimicrobial metal layer can be by electroless deposition, for example. The electroless deposition can be carried out using a copper salt source material which in solution is reduced to metallic copper in the presence of a reducing agent which in turn gets oxidized and the metallic copper atoms are deposited on any surface in the bath including the device surfaces to generate a copper or copper alloy antimicrobial metal layer. The method can further include pretreating the common touch surface of the polymeric material by a preliminary step of activating the polymer surface, e.g., introducing a surface roughness by, for example, chemical etching, mechanical abrasion, physical etching, and/or plasma treatment.

When discussing the polymeric common touch surfaces and metal coatings applied thereto in the form of an antimicrobial device, or in the context of a method herein, relative details from a discussion of either is considered applicable to the other, whether or not they are explicitly discussed in the context of that example. Thus, for example, in discussing a bicycle handle or grip in the context of the device, such disclosure is also related to any methods and/or systems also included herein, and vice versa, etc. Furthermore, the term “antimicrobial device” can be a fully assembled device, such as a bicycle, or can be a part of a larger device, such as a bicycle grip that can be attached to a bicycle handle bar. In either case, the common touch surface in this example may include the portion of the grip that touches the hand when in use, whether installed on a bicycle or not.

The term “common touch surface” herein is defined to include any surface of an object or device that is specifically designed or configured to be touched at a specific location, often by multiple individuals if used in a capacity for multiple users to touch, e.g., hand grip on a bicycle that is for rent or for multiple people to use or even by a single user with multiple uses without cleaning between events. Thus, not all touchable surfaces are considered to be “common touch surfaces” as defined herein. For example, a vinyl window frame would not generally be considered to be a common touch surface, but a plastic locking handle mechanism and a raised handle area where a window is touched to open and close the window would be considered to be a common touch surface. As another slightly more complicated example, a bicycle can technically be touched at any location, but common touch surfaces of a bicycle would include only areas where a user interfaces with the bicycle during normal use (not repair or assembly). Examples include pedals, seats, hand grips, gear shifter handles, bells, water bottles held by the frame, bike locks, bike racks or baskets installed to carry items, tire inflation stems, etc. Rubber tires, the general frame body (unless there is a carrying handle or location), tire spokes, gears, cables, chains to actuate gears, etc., would not be considered to be common touch surfaces. Additionally, common touch surfaces as defined herein include any surface of a device intended to contact intact skin of a human in the normal course of use, where multiple people when using the device normally would touch the device at the same location in the same way. Thus, a medical device implanted or surgically installed (into or through the skin) would not be considered to include a common touch surface because the surgeon touches the device to install across the skin (which is not intact skin) and the surgeon uses his or her hands to do so. On the other hand, the patient passively touches that same object, perhaps even at the same location on the device, or may even touch the device with his or her hands to adjust the device, but does so differently than the surgeon did when installing the device. Thus, in one case, one person touches the object with his or her hands and the other person touches it where it is used or installed or may adjust it with his or her hands, but does not interact with the device in the same way as the surgeon who cuts the skin and implants the device. Such a device would not be considered to have a common touch surface in regular use. However, there may be medical equipment that is used in hospital rooms, doctor's offices, dental offices, etc., that does get used commonly by the medical professional and the user, e.g., sink handles, bed rails, computer keyboards, walkers, wheelchairs, crutches, etc. Those would be considered to have common touch surfaces.

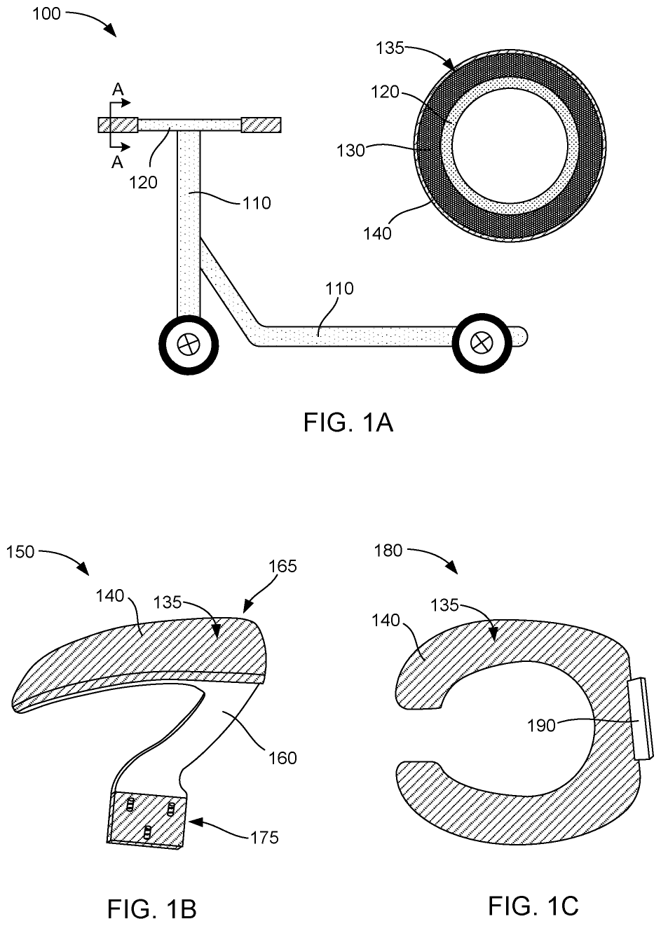

Referring now to FIGS. 1A-1C, a few non-limiting example devices are shown that each include a common touch surface that may be repetitively touched by multiple individuals at the same location and in the same way, particularly if the device is one that is rented out, e.g., by the ride, by the minute, by the hour, by the day, etc., or commonly used by multiple people in a sharing arrangement or situation, e.g., chair, public (or even private) bathroom, etc.

In the particular example shown in FIG. 1A, the device is a scooter 100 (or the device could be the hand grip in some examples, such as a hand grip sold separately to apply to a scooter or other device), such as an electric scooter rented out on a per-ride or per-minute basis by companies such as Bird®, Lime®, Skip®, Scoot®, Spin®, or other similar companies. The scooter includes a scooter body 110 with a handle bar 120. The handle bar may include a hollow tube, for example, but could be of any construction, material, or configuration suitable for steering the scooter (or the bicycle, the motorcycle, the Vespa®-like scooter, the Segway®, etc.). In the example shown, the handle bar includes handle bar grips, or hand grips 130, which include a common touch surface 135. The handle bar and the hand grips are shown in cross-section as well, with the location and direction of view illustrated at section A-A. The hand grips are typically constructed of a polymer material, such as plastic, rubber, or other polymeric material. An antimicrobial metal layer 140 is applied to the common touch surface of the hand grips in this example, as shown in this example as a thin layer (or layers) of the antimicrobial metal. Note that in this and other examples described herein, the common touch surface is coated or plated by the antimicrobial metal layer, and thus, the user does not touch the “common touch surface” per se. Rather, the surface of the polymer is referred to as the common touch surface as that is the surface that the user typically would contact, were it not for the antimicrobial metal layer providing an antimicrobial barrier between the user and the polymer surface. The antimicrobial metal layer 140 can be or can include, for example, copper, silver, zinc, gold, or an alloy thereof, and can be in the form of an elemental metal or combination of elemental metals as an alloy, or even as a combination of elemental metal(s) and a non-metal as an alloy metal/non-metal alloy. In one example, the thin metal layer is copper or a copper alloy. The thin metal layer can be as described herein with respect to material, layer thickness, application process, etc.

Referring now to the example shown in FIG. 1B, the device shown is an arm rest 150, such as for a chair, e.g., office chair. The arm rest includes a structural support 160 (or substrate) that can be of any material, e.g., polymer (plastic, rubber, etc.), metal, wood, ceramic, carbon fiber, etc. In the example shown, the upper portion 165 of the structural support is where the common touch surface 135 may reside, which may include a non-metallic surface, e.g., polymer, leather, wood, ceramic, etc. An antimicrobial metal layer 140, as shown, is applied to the common touch surface of the arm rest in this example. In one example, the structural support may be non-metallic and the antimicrobial metal layer may be applied directly to the upper portion which is also of the non-metallic material. In another example, such as when the structural support is metal, the upper portion may be coated or covered with a non-metallic material which is then coated by the anti-microbial metal layer. These and other arrangements can be implemented in accordance with the teachings herein. In further detail, in this example, there is also shown a second (lower) portion 175 of the arm rest that may also be coated. This second portion is not considered to be a common touch area (as it would only be regularly touched during assembly and not during normal use). However, this example is provided to illustrate that the present disclosure is not limited solely to the coating of common touch areas, as other areas may be coated in addition to the common touch area for any of a number of reasons, e.g., decorative, strength, convenience in manufacturing, etc.

Referring now to the example shown in FIG. 1C, the device shown is a toilet seat 180, which is part of a toilet that is commonly touched by multiple users. The top of the toilet seat can be a non-metallic material, such as polymer (plastic, rubber, Styrofoam, etc.), metal, wood, ceramic, porcelain, carbon fiber, etc. In the example shown, the upward-facing portion (when in position to use) of the structural support is where a common touch surface 135 may reside, which may include a non-metallic surface, e.g., polymer, leather, wood, ceramic, etc. In the example shown, an antimicrobial metal layer 140 is applied to the common touch surface. However, in further detail, the bottom or underside of the toilet seat may also be partially or fully coated (not shown), as the bottom of the seat may be the only location where some users would touch with their hand, such as when lifting or lowering the seat for use. Thus, the upper surface (as shown) is considered to be a common touch surface because a user would touch that surface while sitting. Furthermore, the underside surface (not shown) of the toilet seat would also be considered to include a common touch surface, as that surface may also be touched by the hand while lifting or lowering the seat for use. Thus, the underside may also be fully or partially coated with the antimicrobial metal layer. Furthermore, in this specific example, an attachment location 190 is shown where the toilet seat is attached to the toilet. In this example, since this is not a common touch surface, it might not be coated with the antimicrobial metal layer, as shown.

As a note, with the examples shown in FIGS. 1A-1C, and any other example described herein, the application of an antimicrobial metal layer to a non-metallic common touch surface can provide antimicrobial benefits as described. However, there is no requirement that all common touch surfaces of a device be coated as described herein, as long as at least one non-metallic common touch surface is coated on the device. Coating multiple or all of the common touch surfaces, or additionally coating areas that are not common touch surfaces or which are metal, would be the choice of the designer, such as may occur after a cost-benefit analysis or for some other reason.

Turning now to more detail regarding the antimicrobial metal that can be applied to or electroplated on the polymeric common touch surfaces as described herein, examples of metals that can be used include copper, silver, zinc, gold, or alloys thereof. The term “alloys” include various combinations of two or more of copper, silver, zinc, or gold, but can also include alloys of one or more of these metals with any other metal(s) or non-metal(s) that may provide a therapeutic or other practical property. For example, as copper oxidizes, copper can be alloyed with another metal, such as silver, zinc, and/or gold, but may also be alloyed with metals that may not be necessarily antimicrobial in nature. Examples of other metals that can be alloyed with copper include iron, nickel, aluminum, etc., for the purpose of slowing or preventing oxidation, or for some other therapeutic or practical purpose, e.g., enhanced metal adherence to the medical device surface material, modification of metallurgic properties such as flexibility and/or resilience, etc. As copper is a good metal for providing anti-infective properties due to its ability to contact kill without ion diffusion into or around neighboring tissue, if copper is used, it can be included in the alloy as a substantial portion, e.g., greater than 50 wt %, and a lesser proportion of other metals (or non-metals) may be included to contribute to reduction in copper oxidation, to contribute to antimicrobial effect, e.g., silver, zinc, and/or gold, to contribute to another therapeutic effect, to address a manufacturing concern, to enhance or improve a metal alloy physical property, e.g., flexibility, resilience, malleability, medical device adhesion, etc. Thus, if a copper alloy is used, in one example, the copper can be present in the alloy at from 50 wt % to 99 wt %, from 50 wt % to 95 wt %, from 50 wt % to 90 wt %, from 50 wt % to 80 wt %, from 55 wt % to 99 wt %, from 55 wt % to 95 wt %, from 55 wt % to 90 wt %, from 55 wt % to 80 wt %, from 60 wt % to 99 wt %, from 60 wt % to 95 wt %, from 60 wt % to 90 wt %, from 60 wt % to 80 wt %, or from 55 wt % to 70 wt %.

In some examples, the antimicrobial metal layers applied to polymeric common touch surfaces can be further treated with a protective coating (over the metal surface).

Examples of such protective coatings may include wax, hydrophilic/hydrophobic material, etc. to prevent corrosion.

A metal-based electroplating/electroless deposition technology used to coat polymeric common touch surfaces can address the persistent problem of infections or the transmission of communicable diseases from person to person. These coatings can be designed, for example, to adhere well to the polymeric common touch surface, be applied as a thin and thus relatively inexpensive coating which may retain some of the flexibility of the underlying polymer in some instances, and have long-term and broad spectrum efficacy without relying on release/elution of an active agent in some instances, e.g., copper or copper alloy as a contact killing metal. That being stated, electroplating or sputter coating of elemental metal may be used in combination with other antimicrobial impregnation or solution applications with additional benefit in some instances.

In more detail with respect to copper, in addition to the cationic nature of copper ions (Cu²⁺) that cause them to bind or become attracted to negatively charged protective cell wall components and obliterate or otherwise disrupt or damage the cell membrane or wall, e.g., bacteria or other microbes, copper can also kill microbes by contact with the elemental metal or with an alloy of the elemental metal (rather than by ions that slowly diffuse into solution over time). Thus, copper in particular can be a good material for use in the coating on a polymeric common touch surface because it can kill pathogens continuously where the metal is directly interfacing with the microbes to actively reduce microbial colonization at the interface, regardless of ion diffusion. This can occur by virtue of the charge density where the microbe contacts the copper or copper alloy, which may cause membrane damage, nucleic acid damage, and/or generation of a reactive oxygen species that may be detrimental to the microbe. Thus, copper ions do not necessarily need to diffuse or leach into the microbes to be effective, though some diffusion around the common touch surface can provide an added area of protection over time (the time it takes to diffuse outward). Instead, the native elemental or alloyed copper surface can possess active antimicrobial properties that can prevent colonization. Because of the contact killing nature of copper in particular, elemental or alloyed copper coatings last long-term without depletion or consumption of the killing effectiveness. In still further detail, an additional benefit of using a metal with contact killing properties, e.g., copper, is that it may not expose the subject who contacts the surface with undesired high dosages of copper.

The use of a thin coating of copper for use on a polymeric common touch surface may not have been considered previously as an antimicrobial candidate for coating for some softer or flexible common touch surface because with common touch surfaces, there may be a desired degree of flexibility or softness that may be desirable. A substantially thick copper coating, or even a polymeric material compounded with high concentrations of copper salts in the resin, can become quite rigid. On the other hand, a thin copper coating may not cause the polymeric common touch surface to be overly rigid in some cases due to the very thin layer of metal that can be applied by electroless deposition or electroplating. Even though applied very thin, copper does not rely on elution for killing power against many pathogens, and thus, will last a long period of time without excessive worry of depletion, and furthermore, can be prepared so that it does not flake or readily flake off of the non-metallic surface. With this in mind, combinations of electroplated layers of contact killing metal(s) with antimicrobial eluting compositions (e.g., impregnated material, deposited particulate material such as metal salts, etc.) can sometimes provide additional antimicrobial activity. Furthermore, in some instances, the use of antimicrobial metals, such as copper or copper alloy, can be attractive. By coating various parts with antimicrobial metal, the look of this material can be achieved without the cost associated with building the entire part or device out of the antimicrobial metal, for example.

Thus, in accordance with examples of the present disclosure, an antimicrobial metal can be coated as a thin layer(s) to a polymeric common touch surface, e.g., from a single atom of thickness to 1 mm, from 0.0001 μm to 50 μm, from 0.001 μm to 1 μm, or from 0.01 μm to 0.1 μm, etc., to provide an antimicrobial layer thereon. The “single atom” can be based on the size of the metal atom of the antimicrobial metal, or if an alloy, the average size of the atoms present in the alloy, for example. The metal can be as described previously, and can include copper, silver, zinc, gold, or an alloy thereof. In one example, the metal can include copper, which is a contact killer. In still further detail, the metal coating can be a copper or copper alloy layer on a polymeric common touch surface and can be considered to be effectively non-leaching. Even as non-leaching materials, they can retain their contact killing properties over a long period of time, e.g., 3 months or longer, 6 months or longer, 1 year or longer.

As mentioned above, and in further detail hereinafter, the antimicrobial metal can be applied as a layer by any of a number of methods, such as sputter coating, spray coating, or dip coating. However, in one specific example, the antimicrobial metal layer can be applied by electroless deposition. With this process, continuous layers of atomic metal, such as copper or copper alloy, can be electrically applied on a polymeric common touch surface that may come into contact with multiple humans or even humans and animals. The thin layer can be applied so that the polymeric common touch surface, where desired, can retain some of its flexibility. In other examples, the thickness can be such that the polymeric common touch surface becomes more rigid. There may be occasions to select thicker, more rigid coatings, and other occasions to select thinner coatings, depending on the application. For example, a soft hand grip may be a place where a thinner coating may be desirable so that some of the give of the underlying material can be retained.

In one example, to achieve a non-leaching layer of metal, such as a copper or copper alloy layer, the metal layer can be applied to a polymeric common touch surface by electroless deposition in some examples. For example, the technology may involve electrically depositing a continuous layer or layers of atomic copper over an existing polymeric surface. Plastics, rubbers, and other polymer surfaces that can be electroplated using this technology include, but are not limited to, acrylonitrile butadiene styrene (ABS), polypropylene (PP), polystyrene (PS), polyethylene (PE), polyvinyl chloride (PVC) polycarbonate (PC), polyurethane (PU), silicone, polyethylene terephthalate (PET), fluoropolymer, rubber, etc.

As mentioned, electroless deposition chemistry allows electrically inert substrates, such as plastics, rubbers, or other polymers, to be plated with conductive metals facilitated by in-solution electrochemistry. An example of an electroless deposition process that can be used to apply a thin metal layer to a non-metallic surface, such as a polymeric surface, can be prepared or “activated” for application of the antimicrobial metal. Preparation can include physical preparation and/or chemical preparation. For example, the polymeric common touch surface can be configured or modified to include a surface that makes it even more adherent to the metal layer applied thereto. Surface modification can be introduced by cleaning, washing, etching (e.g., chemical, mechanical, physical), chemical preparation (e.g., alkaline wash, plasma treatment, etc.), roughing (e.g., sand or particle blasting), molding, shaping, etc.

After surface preparation, a metal-plating bath can be prepared for applying a thin layer of the anti-microbial metal or metal alloy. Using copper electroplating as an example, Formula I shows an example chemical equation for electroplating a non-conductive material, as follows:

CuY²⁻+2CH₂O+4OH⁻→Cu⁰+H₂+2HCOO⁻+Y⁴⁻+2H₂O Formula I

where Y represents a copper anion, such as sulfate, chloride, or phosphate; and Cu⁰ represents elemental copper electroplated on the surface of the substrate being plated. At a thin enough layer, the metal coating applied by electroless deposition may, in some instances, retain some of the physical features of the underlying polymeric common touch surface, e.g., a textured hand grips may retain some of its texture even with the metal layer applied thereto. Thus, grip profiles, branding molded into the hand grips, and in some instances, some of the softness or give, can be retained, even with the antimicrobial metal applied thereto.

To apply, the polymeric substrate can be dipped in a bath of a divalent metal, such as copper or other antimicrobial metal or alloy, for example, to give it a very thin coating of electrically conducting metal (typically less than 1 μm). As mentioned, there are other application techniques, such as dipping, sputtering, and/or spraying methodologies that can be used to establish thicker layers, such as by multiple dip coatings or multi-coat spraying layers so that the antimicrobial metal “layer” is actually multiple layers of metal. Thus, the term “layer” includes any arrangement of metals as a single atomic layer, or as a single metallic layer with multiple atoms in thickness, or as multiple adjacent metallic layers that function as a unitary or composite “layer” of metal. With this in mind, if electroplating a second layer after a first electroplated layer is applied directly to the polymeric common touch surface, the previously applied metal can be electroplated to form a second coating using more conventional methods where a metal base is electroplated, e.g., with a metal ion source and a charge carrier while applying electrical potential to the charge carrier bath. Depending on the physical properties of the antimicrobial metal layer desired, the electroplating can occur leaving a layer that is less susceptible to becoming damaged by wear and tear that the plated part has to withstand. Furthermore, copper in particular is considered to be essential nutrient that is generally considered to be non-toxic when contacted by the skin, as evidenced by the long use of copper watches, copper jewelry, etc.

By using electroplating/electroless deposition to apply an antimicrobial metal to a polymeric common touch surface, there can be several advantages achieved. For example, the electroplating can be carried out so that most or all of the elemental metal from the metal source can be made available to the surface of the substrate to impart a higher degree of killing power, and in the case of copper, a higher degree of contact killing potential. This can also help bind the metal tightly to the substrate, even to a polymeric substrate or other non-conductive substrate with negligible leaching.

Electroless deposition/electroplating can also provide for the application of a more precisely controlled application of the thin metal layer, which enables surface modification without significantly compromising device features, e.g. flexibility, comfort, etc. The nature of electroless chemistry incorporates layers of the metal on an atom-by-atom level with areas exposed getting coated, including deformities, cracks, crevices, etc. This can prevent bacterial colonization at the surface, rather than permit colonization followed by killing through ion diffusion. These types of metal layers can also provide a consistent cover of substrate surfaces that may be inherently non-uniform. Furthermore, initial investigations have indicated that electroplated metals seem to resist flaking under mechanical stresses including tensile and bending forces. The nature of electroless chemistry incorporates layers of metal atom-by-atom onto an exposed surface of the polymer substrate, which includes its deformities, cracks, and crevices, for example. Because of this, a consistent coverage of a non-uniform surface can be achieved. Furthermore, another difference between the use of electroplating rather than impregnation or chemical solution coating technologies is that electroplating provides a new common touch surface to be contacted by the user rather than a modified common touch surface, e.g., a metal salt deposited as particulates on a surface or impregnated within the material near the surface, etc. With a new metallic surface applied, microbial colonization may be less likely, particularly if the metal includes a contact killing metal, such as copper. With solution coatings or impregnation, those systems may rely more on ionic diffusions or elution from the common touch surface, likely offering less protection. On the other hand, electroless deposition can be an attractive option of antimicrobial metal application, as it can be cheaper than other methods where a layer of such a metal is applied, e.g., sputter coating. That stated, in addition to electroless deposition, these or other application process can be implemented in accordance with the present disclosure in some examples.

In further detail, upon removal of the polymeric substrate with newly applied electroplated metal from the metal-plating bath, the electroplated part can then be dried by any of a number of methods, such as with ambient air drying, forced air drying, heat drying, insert gases, or the like.

There are many types of structures with common touch surfaces that may be coated with the antimicrobial metal layers of the present disclosure, but which are not specifically shown in the FIGS. Thus, these common touch surfaces can likewise be coated with a thin antimicrobial metal layer, such as copper, silver, zinc, gold, or an alloy thereof to reduce the impact or chances that a subject will pass along or a subject will receive contact with a live bacterial colony. The thin metal layer can be as described previously with respect to material, layer thickness, application process, etc.

The example shown in FIGS. 1A-1C are drawn to various devices with non-metallic touch surfaces, e.g., hand grip, arm rest, toilet seat, but it is understood that these are but a few examples of non-metallic common touch surface that can colonize and/or transmit bacteria from one human host to another. Other example common touch surfaces of polymer material can be coated or plated with an elemental metal or elemental metal alloy to provide an antimicrobial effect. As an example, the common touch surface can be on a kitchen, bathroom, or laundry room device, and in some examples may include a countertop, a tabletop, a cutting surface of a cutting board, a utensil handle, a toilet seat, a plumbing actuator, a cabinet handle, an appliance actuator, a toothbrush handle, a hairbrush or comb handle, a drinking fountain actuator, a handicap hand rail or support handle, a bathroom door handle or know, a tap or faucet, or a chair or stool. The common touch surface can be on a financial instrument or financial device, and in some examples may include a credit card, a region surrounding an opening for inserting or receiving credit cards or cash, compartments in purses and wallets, or buttons on pay stations. The common touch surface can be on a sporting or non-electronics game play equipment, and in some examples may include a hiking or ski pole handle or hand grip, a bowling ball finger hole, a life vest fastener, a snorkel mouthpiece, a scuba regulator mouthpiece, a handle or hand grip on gym equipment, a weight stack pin, a sporting equipment seat, a racquet handle, a bat handle, a club handle, a ball, a pool cue handle, a paddle handle, a flag handle, a gun or starter pistol handle, a jump rope handle, an oar handle, a fencing handle, an archery handle, an equestrian equipment handle, board game pieces, dice, cards, a gaming table arm support, a poker chip, a racket grip, swimming goggles, a shoe insole, or a sweat band. The common touch surface can be on transportation equipment, and in some examples may include a steering wheel, a hand break, a gear shifter handle, an arm rest, a cup holder, a control surface actuator, a key, a key fob, a passenger tray table (e.g., airline or train food tray table), a door handle or knob, a bicycle hand grip, a motorcycle or moped hand grip, a scooter hand grip, a self-balancing transporter hand grip, a public transportation handhold, a luggage handle, a luggage fastener, an elevator button, a baggage cart, or a head rest. The common touch surface can be on an electronics device, and in some examples may include a keyboard button, a mouse button, a computer monitor control, a television control, a printer control, a scanner control, an audio system control, a remote control, a tablet casing, a cellular phone casing, a protective case for a tablet or a cellular phone, a landline hand piece, a landline control, a watch control, a watch band, a laptop or desktop control, a laptop casing, a slot machine handle, a gaming controller handle, a gaming controller control surface, a joystick, a button on a cellular device, a laser pointer, or a console device. The common touch surface can be on a child use device, and in some examples may include a toy handling surface, a playground equipment handle or rail, a sippy cup handle, a bottle handle or grip, a high chair food tray, a stroller handle, a stroller food tray, a stroller toy bar, a stroller arm rest, a car seat handle, a car seat arm rest, a crib railing, a swing tray, a swing seat, a bouncer handle, a book cover, a rattle handle, a rattle, a teething handle, a diaper disposal cover, a diaper disposal handle, a diaper bag handle, a baby wipe lid, a diaper changing station, a pacifier handle, or a rocker. The common touch surfaces may likewise be for common touch surfaces other than those touched by the hand, such as where there may be mucosal touch surfaces, e.g., saliva contact where multiple children, or the same child repeatedly, may contact the same toy with their mouth. The common touch surface can be on a non-implantable health care device, and in some examples may include a medical or dental instrument handle, a bed rail, a countertop at a health care facility, a wheel chair handle, an external ventilator, an arm rest on a physical therapy or dialysis or infusion therapy chair, or a handle on a lighting apparatus in an operating room or clinic. Other examples can also be considered for use at the common touch surfaces.

Essentially, any common touch surface constructed with a non-metallic surface, e.g., polymer, leather, wood, ceramic, porcelain, glass, fabric and textiles, paper, carbon fiber, etc., that can be coated with a thin antimicrobial metal layer, and which may not be regularly cleaned prior to subsequent use as described herein, can benefit from the present technology. Many of these items get touched multiple times a day by different humans in the same way, or by the same human repeatedly, and that repetitive contact can lead to the transfer of bacteria from a first infected host to a second previously uninfected host. As an example, credit cards are touched by multiple people on a daily basis as they are handed back and forth between a customer and a vendor. This brief contact may be enough to transfer bacteria from one host to the next. Likewise, cellular phones, such as smartphones, are passed back and forth between friends regularly, and a moist plastic surface may be a place that carries colonies of bacteria. Coating such surfaces with a thin layer of an antimicrobial metal, such as elemental copper or elemental copper alloy, can provide for microbial contact killing, making passing of credit cards, smartphones, etc., between individuals safer with respect to risk of microbial transmission.

It is to be noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the description herein.

Sizes, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1.0 to 2.0 percent” should be interpreted to include not only the explicitly recited values of about 1.0 percent to about 2.0 percent, but also to include individual values and sub-ranges within the indicated range. Thus, included in this numerical range are individual values such as 1.1, 1.3, and 1.5, and sub-ranges such as from 1.3 to 1.7, 1.0 to 1.5, and from 1.4 to 1.9, etc. This same principle applies to ranges reciting only one numerical value. Furthermore, such an interpretation should apply regardless of the breadth of the range or the characteristics being described.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

It is also noted that any of the device features described herein and/or shown in the FIGS. can be combined together in any manner that is not specifically shown or described. For example, it is not the purpose of the present disclosure to put together every possible combination of features in the drawings or description, but rather to describe fully the combination of concepts to be combined with various types.

### EXAMPLES

The following illustrates several examples of the present disclosure. However, it is to be understood that the following examples are only illustrative of the application of the principles of the present disclosure. Numerous modifications and alternative structures, compositions, methods, and systems may be devised without departing from the present disclosure. The appended claims are intended to cover such modifications and arrangements.

### Example 1—Bacterial Colonization Study of Copper-Plated Silicone and Copper-Plated Fluoropolymer Against Staphylococcus aureus

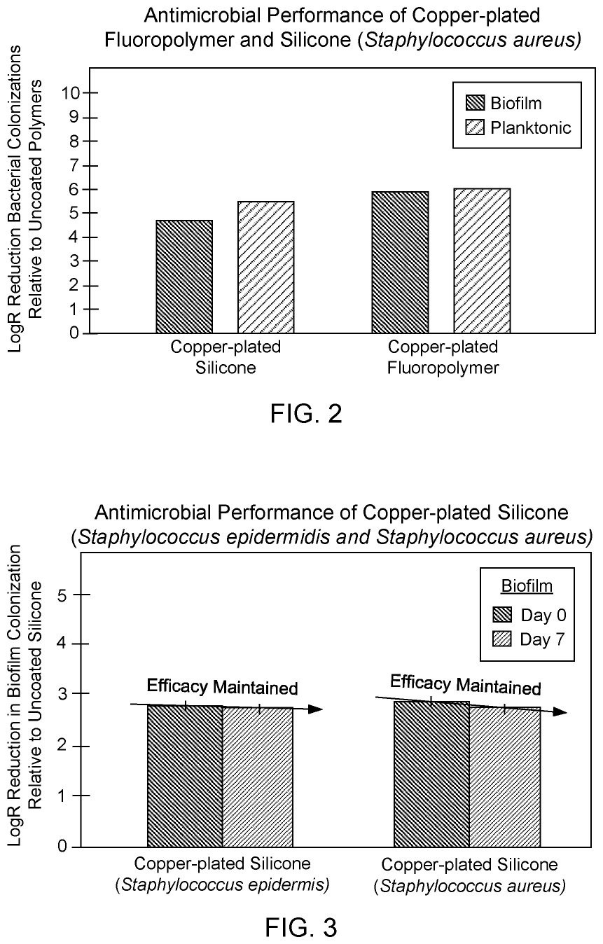

In vitro antimicrobial performance of copper-plated silicone and copper-plated fluoropolymer was compared to uncoated silicone and uncoated fluoropolymer respectively. In further detail, the two different types of copper-plated polymers were characterized for performance based on a Log-scale reduction scale (Log R) compared to their respective uncoated polymer. Bacterial colonization related to the uncoated polymers is not shown, as it merely provides a baseline to report the relative data (Log R) of antimicrobial improvement of the copper-plated polymer compared to the uncoated polymer. Thus, the higher the Log R value reported, the lower the level of bacterial colonization because a high value indicates a higher level of improvement against the uncoated polymer substrate. In this study, a silicone (comparative) and a fluoropolymer (comparative), as well as the copper-plated silicone and fluoropolymer were exposed to Staphylococcus aureus. The groups in this study were exposed to daily saline change-out for 7 days prior to microbial challenge. After 7 days, the various substrates were exposed to a bacterial challenge at a concentration ˜1×10⁴ CFU/mL at 37° C. in in the presence of saline-based nutrient broth for 24 hours. Both biofilm (adherent bacteria) and planktonic (free floating bacteria) recoveries from the respective copper-plated and uncoated polymer materials and surrounding fluid media was collected, and the data is shown in FIG. 2. As can be seen, both the copper-plated silicone and copper-plated fluoropolymer exhibit significant antimicrobial activity relative to the uncoated polymer with respect to biofilm colonization and planktonic recovery.

### Example 2—Bacterial Colonization Study of Copper-Plated Silicone at 0 Days and 7 Days Against Staphylococcus epidermidis and Staphylococcus aureus

In vitro antimicrobial performance of a copper-plated silicone was compared to an uncoated silicone with two different types of bacterial challenge, namely Staphylococcus epidermidis and Staphylococcus aureus. The copper-plated silicone was characterized for performance based on a Log-scale reduction scale (Log R) compared to the uncoated silicon. Bacterial colonization related to the uncoated silicone is not shown, as it merely provides a baseline to report the relative data (Log R) of antimicrobial improvement of the copper-plated silicone compared to the uncoated silicone. Thus, the higher the Log R value reported, the lower the level of bacterial colonization because a high value indicates a higher level of improvement against the uncoated silicone substrate. The data collected in this study was based on a bacterial challenge where a concentration ˜1×10⁶ CFU/mL of one or the other bacteria was exposed to the silicone or the copper-plated silicone, and the colonies were allowed to incubate at 37° C. in in the presence of a saline-based nutrient broth. Biofilm (bacterial adherent) recoveries were collected at Day 0 (initial) and after Day 7 (1 week later). The samples were subjected to daily saline change-out between Day 0 and Day 7. At both time points, the samples were exposed to a 24 hour bacterial challenge. The data collected is shown by way of example in FIG. 3. As can be seen, the copper-plated silicone effectively retained antimicrobial efficacy after 7 days of simulated bacterial exposure conditioning, meaning that the copper-plating continued to be effective against bacteria when other types of antibacterial coatings, such as antibiotics or other types of diffusing materials may have stopped working. This indicates a longer term antimicrobial efficacy of copper-plating, likely due to its contact killing activity against many microbes.

### Example 3—Bacterial Colonization Study of Copper-Plated Soft Polymeric Hand Grips Against Staphylococcus aureus

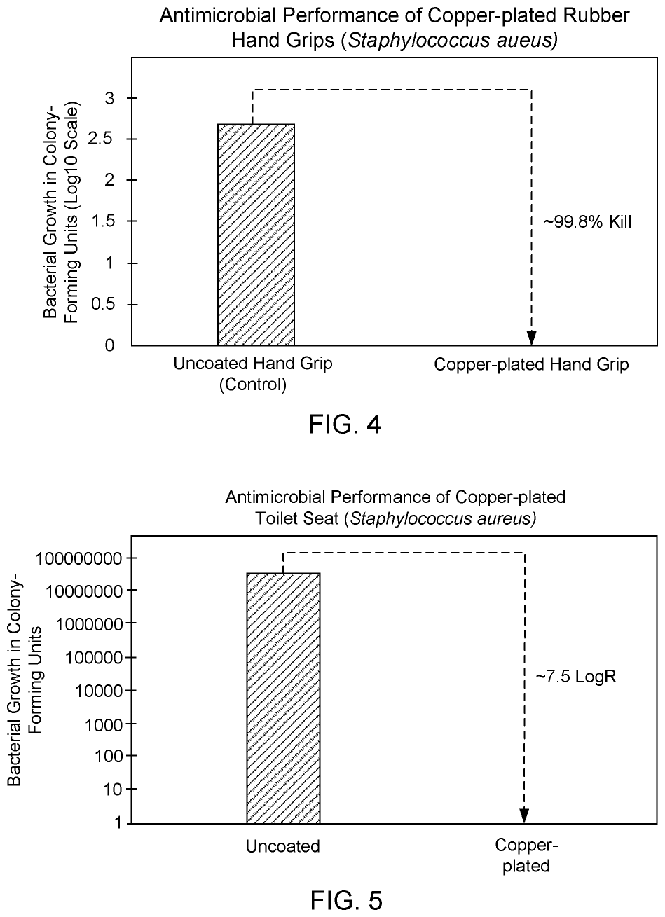

In vitro antimicrobial performance of copper-plated hand grips was studied by preparing hand grip prototypes that would be suitable for use on a scooter, bicycle, handles on sports equipment, weight lifting equipment, or the like. In this study, the copper-plating was applied at a thickness of about 0.1 μm using an electroplating process. Both uncoated hand grips and copper-plated hand grips were subjected to a bacterial challenge. The handgrips were constructed of Durasoft polymer (DSP) material (2-hydroxyethyl 2-methylprop-2-enoate). Specifically, 100 μL of a ˜1×10⁵ CFU/mL inoculum was added to each surface to end up with ˜1×10⁴ CFU challenge and spread evenly to simulate a microbial challenge in the field. After 24 hours, the microbe on each surface was recovered by sonication. The microbial counts were quantitated by serial dilution and plating. The microbial colonization on a surface of the uncoated hand grips as well as on the surface of the copper-plated hand grips was represented as colony forming units on a Log 10 scale). The data collected is provided in FIG. 4, which shows that the copper-plated hand grips demonstrated no growth compared to about 2.5 Log growth on the uncoated hand grip. 2.5 Log growth is approximately the usual microbial load on typical human skin, indicating that the uncoated hand grip received a transfer of bacteria approximating bacteria found naturally on the skin. Thus, it is clear that the copper-plated hand grips had the effect of killing any bacteria that may have been otherwise transferred.

### Example 4—Bacterial Colonization Study of Copper-Plated Toilet Seat Against

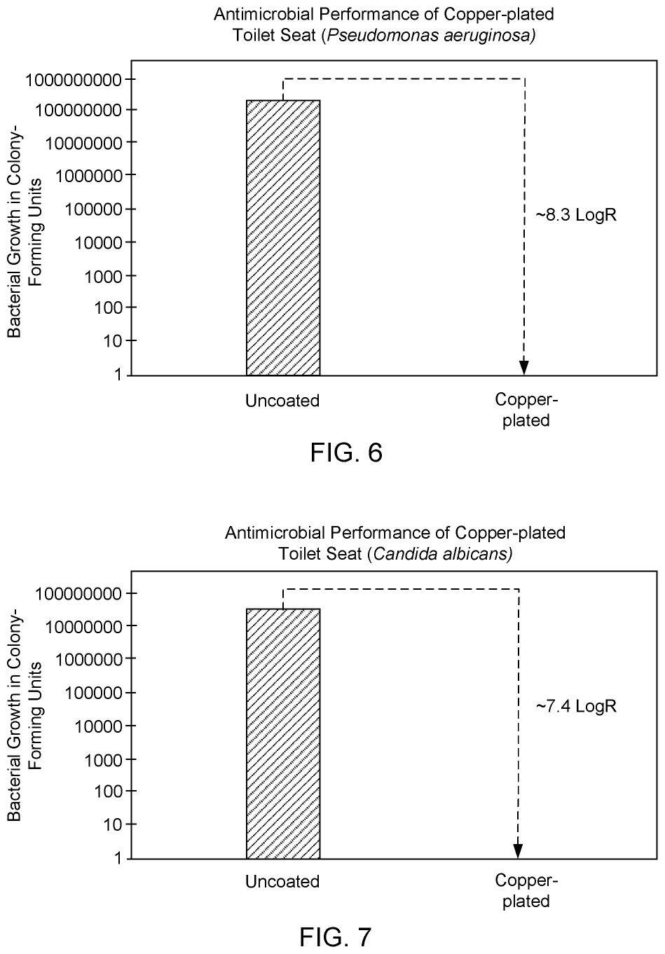

Staphylococcus aureus, Pseudomonas aeruginosa, and Candida albicans In vitro antimicrobial performance of copper-plated toilet seats was studied by comparing antimicrobial performance against uncoated toilet seats. The copper-plated toilet seats were prepared by coating various polypropylene toilet seats with a thin layer of copper using an electroplating process. Both the coated toilet seats and the uncoated toilet seats were studied by exposing both surfaces to common microbial species in the form of droplets for comparison. Three different microbial strains were studied, namely Staphylococcus aureus, Pseudomonas aeruginosa, and Candida albicans. Specifically, in each study, 100 μL of a ˜1×10⁵ CFU/mL inoculum was added to each surface to end up with ˜1×10⁴ CFU challenge and spread evenly to simulate a microbial challenge in the field. After 24 hours, the microbes on the various toilet seat surfaces were recovered by sonication. The microbial counts were quantitated by serial dilution and plating on agar plates. The microbial colonization data from surfaces of the uncoated toilet seats as well as from surfaces of the copper-plated toilet seats was collected, as shown using “Colony Forming Units” represented in FIGS. 5-7. As can be seen, regardless of the microbe chosen for study, the copper-plated toilet seats demonstrated essentially no growth compared to about (˜) 7.5 to 8.3 Log growth on the uncoated toilet seat surface which translates to ˜7.5 to 8.3 Log reduction.

While the forgoing examples and descriptions are illustrative of the principles of the present technology in one or more particular applications, it will be apparent to those of ordinary skill in the art that numerous modifications in form, usage, and details of implementation can be made without the exercise of inventive faculty, and without departing from the principles and concepts of this technology. Accordingly, it is not intended that the technology be unduly limited.

1. An antimicrobial device, comprising:
a common touch surface of a non-metallic material; and
an antimicrobial metal layer applied to the common touch surface at an average thickness ranging from a single metal atom in thickness to 1 mm.

2. The antimicrobial device of claim 1, wherein the antimicrobial metal layer is not applied to areas of the device that are not at the common touch surface.

3. The antimicrobial device of claim 1, wherein the antimicrobial metal layer is an electroless deposition of an elemental metal layer or an electroless deposition of metal alloy layer positioned on the common touch surface.

4. The antimicrobial device of claim 1, wherein the antimicrobial metal layer comprises elemental copper, elemental silver, elemental zinc, elemental gold, or an alloy thereof.

5. The antimicrobial device of claim 1, wherein the antimicrobial metal layer is elemental copper or an alloy of elemental copper.

6. The antimicrobial device of claim 1, wherein the common touch surface is on a public use device.

7. The antimicrobial device of claim 1, wherein the common touch surface is on a hand grip, hand hold, or a hand actuator, and the common touch surface includes a location designed for repeatable contacted by a hand of multiple hosts.

8. The antimicrobial device of claim 1, wherein the common touch surface is on a kitchen device, a bathroom device, a laundry room device, a countertop, a tabletop, a cutting surface of a cutting board, a utensil handle, a toilet seat, a plumbing actuator, a cabinet handle, an appliance actuator, a toothbrush handle, a hairbrush or comb handle, a drinking fountain actuator, a handicap hand rail or support handle, a bathroom door handle, a bathroom door knob, a tap or faucet, or a chair or stool.

9. (canceled)

10. The antimicrobial device of claim 1, wherein the common touch surface is on a financial instrument or financial device.

11. (canceled)

12. The antimicrobial device of claim 1, wherein the common touch surface is on sporting or non-electronics game play equipment.

13. (canceled)

14. The antimicrobial device of claim 1, wherein the common touch surface is on transportation equipment.

15. (canceled)

16. The antimicrobial device of claim 1, wherein the common touch surface is on an electronics device.

17. (canceled)

18. The antimicrobial device of claim 1, wherein the common touch surface is on a child use device.

19. (canceled)

20. The antimicrobial device of claim 1, wherein the common touch surface is on a non-implantable heath care device.

21. (canceled)

22. The antimicrobial device of claim 1, wherein the common touch surface is a surface that regularly comes into contact with a mouth or saliva in use.

23. The antimicrobial device of claim 1, wherein the common touch surface is a surface that regularly comes into contact with a skin surface other than the hand when in use.

24. The antimicrobial device of claim 1, wherein the antimicrobial metal layer is an electroplated metal having a thickness from 0.0001 μm to 50 μm.

25. (canceled)

26. A method of reducing the spread of microbes from host to host, comprising:
providing an antimicrobial device including a common touch surface of a non-metallic material having an antimicrobial metal layer applied to the common touch surface at an average thickness ranging from a single metal atom in thickness to 1 mm;
using the antimicrobial device with a first host coming into skin contact with the common touch surface;
subsequently using the antimicrobial device with a second host coming into skin contact with the common touch surface at a later point in time, wherein the second host is the same host or is a different host than the first host,
wherein in between the first hosing using the antimicrobial device and the second hosting subsequently using the antimicrobial device, the antimicrobial metal layer kills a portion of the microbes thereon left by the skin contact by the first host.

27. The method of claim 26, where the second host is different than the first host.

28. (canceled)

29. The method of claim 26, wherein the antimicrobial device includes common touch surface with an interface for skin contact using the hand, and wherein the skin contact by the first user is using the hand and the skin contact by the second user is also by the hand.

30. The method of claim 29, wherein the antimicrobial device is include on rental device, and the common touch surface is included as part of a hand grip, a hand actuator, a hand hold, or a hand rest: or wherein the antimicrobial device is included on a public transportation device, and the common touch surface is included as part of a hand grip, a hand actuator, a hand hold, or a hand rest.

31. (canceled)

32. A method of manufacturing an antimicrobial device, comprising applying an antimicrobial metal layer at an average thickness ranging from a single metal atom in thickness to 1 mm to a common touch surface of a non-metallic material to form an antimicrobial device.

33. The method of claim 32, wherein applying the antimicrobial metal layer is by electroless deposition.

34. The method of claim 33, wherein the electroless deposition is carried out using a copper salt source material which in solution is reduced to metallic copper in the presence of a reducing agent which in turn gets oxidized and the metallic copper atoms are deposited on any surface in the bath including the medical device surfaces to generate a copper or copper alloy antimicrobial metal layer.

35. The method of claim 32, further comprising pretreating the common touch surface of the non-metallic material by a preliminary step of activating a surface of the non-metallic material, wherein activating the non-metallic material includes chemical etching, mechanical abrasion, physical etching, or plasma treatment.

36. The method of claim 35, wherein the non-metallic material includes a polymer.
